(19) United States
(12) Patent Application Publication
(10) Pub. No.: US 2017/0362557 A1
(43) Pub. Date: Dec. 21, 2017

(54) SYSTEMS, DEVICES, KITS AND METHODS FOR SEEDING CELLS OR SETS OF MOLECULES IN AN ARRAY ON A SUBSTRATE

(71) Applicant: NOVELLUSDX LTD., Jerusalem (IL)

(72) Inventors: Yehoshua SHEINMAN, Ra'anana (IL); Revital SHARIVKIN, Ramat-Gan (IL); Merav BELENKOVICH, Jerusalem (IL)

(21) Appl. No.: 15/536,584

(22) PCT Filed: Dec. 15, 2015
(86) PCT No.: PCT/IL2015/051216

§ 371 Date: Jun. 15, 2017

Related U.S. Application Data
(60) Provisional application No. 62/091,669, filed on Dec. 15, 2014.

(30) Foreign Application Priority Data
Dec. 15, 2015 (IL) PCT/IL2015/051216

(51) Int. Cl. C12M 1/32; C12M 1/18; C40B 60/08; C12M 3/00; C12M 1/26

(52) U.S. Cl. CPC C12M 33/06; C12M 3/00; C12M 33/14; C40B 60/08; C12M 1/18; B01J 2219/00621; B01J 2219/00619; B01J 2219/00369; B01J 2219/00722; B01J 2219/00725; B01J 2219/00351; B01J 2219/00743; B01J 2219/00734

(57) ABSTRACT

The present disclosure provides systems, devices and methods for seeding cells or sets of molecules on a substrate by utilizing a seeding mesh, to obtain an essentially homogenous patterned seeding of the cells or sets of molecules on the mesh.

### FIELD OF THE INVENTION

The present disclosure generally relates to the field of providing predetermined arrays of molecules or cell seeding in a pattern on a substrate.

### BACKGROUND OF THE INVENTION

In many fields of scientific research, when large amount of samples or experimental conditions is to be studied, analyzed and compared, high-throughput assays are used, in which large amount of assays can be performed simultaneously. High-throughput assays require dedicated equipment, as well as a platform on which the assays are performed, allowing the identification of each sample. A prominent requirement of a high-throughput assay platform, is maintaining assay parameters as constant or identical as possible between various test points in order to avoid bias. For example, in the field of cellular biology, when cells are the experimental system, a substrate capable of bearing the cells is required. To this aim, plates having multiple wells (multi-well plate), are often used. Using such plates, though considered effective, is not efficient for high-throughput tests, as the number of wells per plate is limited (commonly 12, 24, 48, 96, 384 or 1536 wells per plate) and the volume of reagents used is relatively large. Additionally, the cost of such plates is relatively high. Likewise, performing high-throughput assays on or with various molecules (such as, for example, nucleic acid molecules, proteins, lipids, and the like), requires a dedicated platform capable of effectively bearing such molecules, preferably in an array which allows the identification of each sample. The formation of such dedicated platform is highly time consuming and expensive. Furthermore, in order to reduce bias and experimental artifacts, such platform should provide a high degree of order within and between tested samples, to reduce bias and experimental artifacts. For example, when cells are used as experimental system, homogeneity of cell density in and between different samples is important to reduce background and increase accuracy of the assay and comparison between samples.

There is thus a need in the art for systems, devices, kits and methods for seeding cells or depositing sets of molecules in an array on a substrate, which are high-throughput, simple for manufacturing and use, cost and time efficient and that further allow efficient and homogenic dispersion of the cells or sets of molecules on the substrate, to result in a more accurate, fast and efficient comparison between different parallel assays.

### SUMMARY OF THE INVENTION

The present invention provides systems, devices, kits and methods for seeding cells or sets of molecules in a controlled/predetermined pattern and in an array on a substrate, where the groups of cells or sets of molecules in the array are spatially separated. In some embodiments, the separation between the cells or the sets of molecules in the array is achieved not necessarily by a physical barrier there between, but rather by a space formed between the separate sets of cells or molecules. In further embodiments, the systems, devices, kits and methods for seeding cells or sets of molecules provide a uniform, homogenic and controlled seeding pattern and/or dispersion of the cells or molecules on the substrate. In various embodiments, one or more of the above-described problems have been reduced or eliminated, while other embodiments are directed to other advantages or improvements.

According to some embodiments, there are provided systems, kits, devices and methods for homogeneous seeding of viable cells or depositing sets of molecules on a substrate, using a seeding-mesh. According to some embodiments, seeding viable cells or other molecules to a surface of a substrate using a seeding-mesh results in a cell-seeding pattern or patterns of sets of molecules constrained by structural elements (such as, threads) of the mesh and availed through the holes (apertures) of the mesh. Advantageously, the use of the seeding-mesh for the cell-seeding or depositing of molecules on a surface of a suitable substrate may result in a homogeneous, uniform and/or controllable seeding density having a predetermined seeding pattern. The seeding mesh may further enable the formation of a predetermined array of spatially separated groups of cells or sets of molecules on the substrate. In some embodiments, the spatial separation between the groups of cells or sets of molecules on the substrate is achieved by cell free regions or molecule-free regions, defining the array on the substrate, not by a physical barrier that physically separates between the groups of cells or sets of molecules on the substrate. Advantageously, the use of a seeding-mesh for seeding cells and/or sets of molecules may result in a homogeneous controllable seeding density, while maintaining spatial separation between groups of cells and/or the set of molecules. Thus, the advantageous systems, methods, kits and devices disclosed herein allow the very efficient formation of a predetermined array of cells or sets molecules in a reproducible, accurate, time saving and highly cost effective manner.

According to some embodiments, there is provided a system for seeding cells, the system comprising: a transparent substrate having a cell seeding surface configured for attachment of viable cells; a seeding mesh having a patterned structure, configured to allow passage of viable cells dispensed onto said seeding surface according to a seeding pattern determined by the patterned structure of said mesh; and a cell dispenser, configured to provide viable cells onto said mesh.

In some embodiments, the mesh is made of a polymeric material. In some embodiments, the mesh is made from a polymeric material or other material having low wettability. In some embodiments, the mesh is made of a hydrophobic material. In some embodiments, the mesh is made of a hydrophobic polymeric material. In some embodiments, the mesh may be made of such materials as, but not limited to: nylon, polyester, polyurethane, Polyethylene (PE) polyethylene terephthalate (PET), Polypropylene (PP), Polyvinyl chloride (PVC), Polytetrafluoroethylene (PTFE), Polyvinylidene fluoride (PVDF), Polydimethylsiloxane (PDMS), or combinations thereof. In some exemplary embodiments, the mesh is a nylon mesh. In some embodiments, the mesh is biologically, chemically and/or electrically inert to the cells or molecules deposited therewith or therethrough.

According to some embodiments, the patterned structure of the mesh comprises a weaving lattice pattern or any other desired pattern, determined according to the structure of the mesh.

According to some embodiments, the mesh is configured to enable homogeneous dispersion of viable cells onto said seeding surface such that the cell density is substantially uniform within the perimeters of the seeding pattern on said seeding surface.

In some embodiments, the mesh is configured to enable dispersion of viable cells in a plurality of groups, separated from each other at distinct locations on said seeding surface. In some embodiments, the mesh is configured to enable dispersions of the cell groups in an addressable array. In some embodiments, the cells are maintained in a suitable solution on said seeding surface during and/or after deposition.

In some embodiments, the mesh comprises a grid configured to facilitate separation between said cell groups. In some embodiments, the grid may be integrally formed with said seeding mesh. In some embodiments, the grid is separated from the seeding mesh and is configured to be mounted on the mesh prior to seeding. In some embodiments, the grid comprises a hydrophobic material. In some embodiments, the hydrophobic material is non-toxic to the cells. In some embodiments, the grid may be solid, stiff, or semi-solid when the encountering the cells.

In some embodiments, the system may include a frame for maintaining the orientation of the mesh in alignment with the cells seeding surface. In some embodiments the system may include a frame for positioning/placing/stretching the mesh such that the cells/molecules deposited on it encounter an essentially flat uniform interface. In some embodiments, the system may include a framed container configured to facilitate detaching of said seeding mesh from the seeding surface essentially without affecting the cells. In some embodiments, the framed container may include a flotation means configured to facilitate the detachment of the seeding mesh from the substrate. In some embodiments, a float device/element, if used, may be attached or otherwise be associated with the mesh frame. In some embodiments, the frames may be separate frames or one frame configured to enable one or more of the above mentioned configurations.

In some embodiments, the system may further include a framed container configured to detach said seeding mesh from the seeding surface essentially without affecting the cells. In some embodiments, the mesh is removed from the substrate without affecting the seeding pattern of the cells or otherwise affecting the cells.

According to some embodiments, there is provided a kit for seeding cells, the kit comprising: a transparent substrate having a cell seeding surface configured for attachment of viable cells; a seeding mesh having a patterned structure, configured to allow passage of viable cells onto said seeding surface according to a seeding pattern determined by the patterned structure of said mesh; and a frame for maintaining the orientation of the mesh.

According to some embodiments, the frame is configured to stretch the said seeding mesh such that the cells/molecules deposited on it encounter a substantially flat uniform interface. According to some embodiments, the frame is configured to facilitate detachment of said seeding mesh from the seeding surface essentially without affecting the cells.

According to some embodiments, there is provided a method for seeding cells on a seeding substrate, the method comprising: providing a cell seeding mesh having a patterned structure; mounting the seeding mesh on a surface of a seeding substrate; and dispensing viable cells in a physiologically acceptable medium to the surface of the seeding substrate through the mesh, thereby obtaining multiple viable cells attached to the surface of the substrate according to the pattern of the mesh.

In some embodiments, the method may further include providing a fluid layer between the surface of the substrate and the mesh to facilitate the release of the viable cells from the mesh to the substrate.

In some embodiments, the method may further include separating the mesh from the surface of the substrate without compromising the seeding pattern and/or the vitality of the cells.

In some embodiments, the mesh comprises a hydrophobic grid structured such that upon dispensing the viable cells to the substrate through the mesh, a plurality of viable cell groups are obtained on the surface of the substrate, such that the cell groups are deposited in an array, determined by the grid on said mesh.

In some embodiments, the method may further include a step of incubating the cells before, during or after mounting the mesh on the surface of the substrate.

According to some embodiments, there is provided a system for depositing multiple sets of molecules in a predetermined array on a surface of a substrate, the system comprising: a substrate suitable for attachment of multiple sets of molecules in a predetermined array; a mesh configured to allow passage of multiple sets of the molecules to the substrate, arranged in the predetermined array, such that said sets are separated from each other, wherein said mesh is configured to be approximated to the surface of the substrate and to allow passage of at least some of said molecules onto the surface of the substrate, while maintaining spatial separation of the sets within the designated array; and a dispenser configured to dispense said multiple sets of molecules onto the mesh according to the predetermined array.

According to some embodiments, the sets of molecules are selected from the group consisting of: peptides, proteins, antibodies, enzymes, ligands, nucleic acids, lipids small organic molecules and beads. Each possibility is a separate embodiment.

According to some embodiments, the substrate is deposited with a coating layer conducive to the attachment of the sets of molecules. According to some embodiments, the coating layer is homogeneously coated, deposited on or formed with said surface. In some embodiments, the coating may be selected from, but not limited to: hydrogel, epoxysilane, aldehydesilane, streptavidin, silane, epoxide, maleimide, and the like, or combinations thereof. Each possibility is a separate embodiment.

According to some embodiments, the predetermined array is defined by a grid on said mesh. In some embodiments, the grid is applied by an automated applicator onto said mesh, in a predetermined pattern, said predetermined pattern maintaining spatial separation of the sets within the designated array.

According to some embodiments, the mesh may be removed from the system after the transfer of the molecules to the substrate, without affecting the deposition pattern of the molecules on the substrate.

According to some embodiments, there is provided a kit for depositing multiple sets of molecules in a predetermined array on a surface of a substrate, the kit comprising: a substrate suitable for attachment of multiple sets of molecules in a predetermined array; a mesh configured to carry and/or enable deposition of multiple sets of the molecules arranged in the predetermined array, such that said sets are separated from each other, wherein said mesh is configured to be approximated to the surface of the substrate and to allow passage of at least some of said molecules onto the surface of the substrate, while maintaining spatial separation of the sets within the designated array; and a frame for maintaining the orientation of the mesh and the surface of the substrate.

In some embodiments, the frame may further be configured for stretching the seeding mesh thereon such that the molecules deposited on the mesh encounter a uniform interface. In some embodiments, the frame is configured to promote, enable, allow, facilitate detachment of the mesh from the seeding surface essentially without affecting the deposition pattern of the molecules on the seeding surface or otherwise affecting the molecules deposited thereto.

According to some embodiments, there is provided a method for depositing multiple sets of molecules in a predetermined array on a surface of a substrate, the method comprising: providing a mesh having a patterned structure; mounting the mesh on the surface of the substrate; and dispensing molecules in an acceptable medium to the surface of the substrate through the mesh, thereby obtaining multiple sets of molecules in a predetermined array on the surface of the substrate.

In some embodiments, the method may further include providing a fluid layer between the surface of the substrate and the mesh to facilitate the attachment between the mesh and the substrate and/or the release of the molecules from the mesh to the substrate.

In some embodiments, the method may further include separating the mesh from the surface of the substrate without compromising the deposition pattern of the molecules on the substrate.

In some embodiments, the method may further include a step of providing the surface of the substrate with a first coating layer conducive to the attachment of the sets of molecules. In some embodiments, the first coating layer is homogeneously coated, deposited on or formed with said surface.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions. The following embodiments and aspects thereof are described and illustrated in conjunction with systems, kits, devices and methods which are meant to be exemplary and illustrative, not limiting in scope.

### BRIEF DESCRIPTION OF THE DRAWINGS

Examples illustrative of embodiments are described below with reference to figures attached hereto. In the figures, identical structures, elements or parts that appear in more than one figure are generally labeled with the same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1A schematically illustrates a system for seeding cells or depositing sets of molecules on a substrate, according to some embodiments;

FIG. 1B schematically illustrates a kit for seeding cells or depositing sets of molecules on a substrate, according to some embodiments;

FIG. 1C schematically illustrates an exemplary kit for seeding cells on a substrate, according to some embodiments

FIG. 2A shows an illustration of a perspective view of a framed casing configured to hold a substrate having a surface suitable for attachment of viable cells, according to some embodiments;

FIG. 2B shows an illustration of a top perspective view of a mesh-holding frame configured to hold or stretch a mesh, according to some embodiments;

FIG. 2C shows an illustration of a top view of a substrate casing (holding a substrate) and a mesh-holding frame (holding a mesh), the mesh frame being positioned on top of the substrate casing, according to some embodiments;

FIG. 2D shows a schematic illustration of a top view of a casing with substrate casing (holding a substrate) and a mesh-holding frame (holding a mesh), the mesh frame being positioned on top of the substrate casing, the mesh frame is associated with a float device, according to some embodiments;

FIG. 2E shows a schematic illustration of a cross section view of a casing with substrate casing and a mesh-holding frame (holding a mesh), the mesh frame being associated with a float device, according to some embodiments;

FIGS. 2F-G show illustrations of cross section views of a casing including a substrate and a mesh, according to some embodiments; FIG. 2F—The mesh is in contact with the substrate; FIG. 2G—the mesh is separated from the substrate;

FIG. 3 shows a perspective front view illustration of an exemplary dispenser, according to some embodiments;

FIG. 4A a pictogram showing an example of cells seeded on a substrate, through 100 μm² pores of a nylon mesh thus assuming the mesh's weaving pattern, according to some embodiments;

FIG. 4B a pictogram of a mesh gridded with polymer lines to form an array of 2 mm² chambers;

FIG. 4C a pictogram of cell groups seeded in 2 mm² spot pattern on a substrate using the mesh of FIG. 4B;

FIGS. 5A-C pictograms of cell groups seeded on a substrate surface through a nylon mesh, spatially separated according to a pattern dictated by the mesh's grid; the pattern is maintained after the mesh is removed from the cells. Cell nuclei were labeled with DAPI and the image was acquired under UV lighting, 10× magnification and 20×20 image stitching;

FIG. 6 a pictogram of an exemplary array of antibodies transferred onto a substrate through a suitable gridded mesh.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, the present disclosure provides methods, systems, kits and devices that bring advantageous features for seeding cells or sets of molecules on a substrate. The cells may include any type of cells, including viable cells, fixed cells or cell extracts. The molecules may include any type of molecules including, but not limited to: nucleic acid molecules, lipids, enzymes, ligands, proteins, peptides, antibodies, antigens, small organic molecules, beads, magnetic beads, and the like or any combination thereof. Some of the features include potentially high-throughput, time efficient and low cost seeding of cells or sets of molecules on a substrate for performing different assays simultaneously, while using a smaller area, lower amounts (lower input volume) of cells or molecules as well and lower volumes of subsequent solutions that may be used or required in the process or in downstream manipulations or assays. Other features include a very high accuracy of the assays resulting from the unified homogeneous density of seeded cells or sets of molecules within the array and from the spatial separation between plurality of cell groups or sets of molecules, which may be arranged in an addressable array on the substrate.

According to some exemplary embodiments, the present disclosure provides methods, systems, kits and devices that advantageously allow a cost effective, time saving, versatile and highly efficient and reproducible formation of an array of cells or sets of molecules on a substrate, wherein the cells or sets of molecules may further have density homogeneity within the set. In some embodiments, the characteristics of the array (such as, for example, size, form, shape, thickness, distribution, etc.) may be predetermined at will, in a very cost effective manner. In some embodiments, the groups of cells or sets of molecules are spatially separated on the substrate in an addressable array, allowing use thereof in various downstream high-throughput assays, whereby the homogeneity of distribution of the cells or molecules within the array provides far more accurate and reproducible results.

The following are terms which are used throughout the description and which should be understood in accordance with the various embodiments to mean as follows:

A “cell”, as used herein refers to on any type of cell, of any origin, such as, for example, mammalian and non-mammalian cells, Eukaryotic and Prokaryotic cells or any other type of cells of interest. Exemplary cells can include, for example, but not limited to, of mammalian, avian, insect, yeast, filamentous fungi or plant origin. Non-limiting examples of mammalian cells include human, bovine, ovine, porcine, murine, and rabbit cells. The cell may be a primary cell or a cell line. In some embodiments, the cells may be selected from isolated cells, tissue cultured cells, cell lines, primary cultures, cells obtained from an organism body, cells obtained from a biological sample, and the like. In some embodiments, the cells may be selected from HeLa cells, HEK 293 cells, PC12 cells, U2OS cells NCI60 cell lines (such as, A549, EKVX, T47D, HT29), and the like or combination thereof. Each possibility is a separate embodiment. In some embodiments, the cells are other than osteoprogenitor cells. In some embodiments, the cells may be manipulated cells. In some embodiments, manipulated cells may include, for example, pre-transfected cells, cells transiently and/or stable expressing one or more exogenous genes, and the like. In some embodiments, cells seeded on a substrate are viable cells. In some embodiments, cells seeded on a substrate are not viable. In some embodiments, cell seeded on a substrate may include a cell extract. In some embodiments, the cells are adherent cells. In some embodiments, the term “cell” may further encompass cells in a medium (such as, growth medium), fluid, solution, buffer, serum or other bodily fluids. In some embodiments, the term “seeding” is directed to placing, deploying, dispensing, attaching, adhering, tethering, placing, growing cells on a substrate. In some embodiments, cells may be substantially homogenously seeded/dispersed on the substrate. In some embodiments, the cells may be used for various applications and assays. For example, the cells may be used in biochemical assays (such as, for example, but not limited to: immunostaining, enzymatic reactions, and the like), molecular biology assays (such as, for example, but not limited to: PCR); imaging assays (such as, but not limited to: microscopy (such as, fluorescent microscopy, confocal microscopy, and the like), and the like.

The term “cell group(s)” as used herein may refer to a plurality of cells deployed on a surface of a slide in relatively close approximation. In some embodiments, cells within a cell group are homogenously seeded/dispersed. In some embodiments, a cell group is spatially separated from other cell groups. According to some embodiments a “cell group” may occupy a certain space or a spot or a chamber or a location or on the surface of the substrate. In some embodiments, the groups of cells are spatially separated from each other, wherein the cells in each group are essentially similar. In some embodiments, the seeding pattern and/or density of the cells within and/or between the groups are similar. The groups of cells may be identical, similar or different in composition (type of cells and/or medium), concentration, density, etc. In some embodiments, cell groups are arranged in an array. In some embodiments, the array may be predetermined. In some embodiments, the array may be an addressable array. In some embodiments, the array may be a designated array.

According to some embodiments, the number of cells per cell group is more than about 1*10² cells. In some embodiments, the number of cells per cell group is less than about 5*10⁵ cells.

According to some embodiments, cell density in cell groups is more than about 5*10³ cells/cm² and less than about 5*10⁵ cells/cm².

According to some embodiments, viable cells may include any type of cell, such as, human cell, animal cell, avian cell, plant cell and the like. In some embodiment, the viable cells are adherent cells. In some embodiments, the cells are tissue culture cells. In some embodiments, the cells are tissue-derived cells. In some embodiments, the cells are from a cell line. In some embodiments, the cells are derived from a biological sample of a subject.

The term “molecule” as used herein refers to any type of molecule that may be seeded, deposited, dispensed, and/or delivered to a substrate. In some embodiments, the molecules may be homogenously deposited on the substrate. In some embodiments, the molecules may be artificially synthesized, isolated from a natural source, or both. In some embodiments, the molecules may be selected from, but not limited to: nucleic-acid molecules, proteins, antibodies, enzymes, ligands, antigens, substrates, lipids, small organic molecules, beads, magnetic beads and the like, or combinations thereof. Each possibility is a separate embodiment. In some embodiments, the proteins may include any type of proteins, such as, peptides, enzymes, antibodies, antigens, and the like. In some embodiments, the beads may include any type of beads, at any desired shape, size, form or compositions. For example, the beads may include glass beads, magnetic beads, nanometric beads, polymeric beads, agarose beads, or any suitable beads or modified beads. In some embodiments, the molecules may be arranged (dispersed) in a plurality of sets, separated from each other. In some embodiments, the sets of molecules are spatially separated from each other, wherein the molecules in each sets are essentially similar. In some embodiments, the dispensing pattern and/or density of the molecules within the sets are similar. The sets of molecules may be identical, similar or different in composition, concentration, density, etc. In some embodiments, the molecules may be homogenously dispersed within and/or between sets of molecules.

The term “bead(s)” refer to any type of bead that can be used in biological applications. In some embodiments, the bead may have any globular or spherical shape. In some embodiments, the beads may range in size from nanometric to micrometric size. In some embodiments, the beads may be made of any suitable material. In some embodiments, the beads may be coated with one or more materials, compounds or molecules. In some embodiments, the beads are inert. In some embodiments, the beads are chemically, biologically and/or electrically inert. In some embodiments, the beads are glass beads, metal beads, polymeric beads, magnetic beads, and the like.

The terms “polypeptide,” “peptide” and “protein” are used interchangeably herein to refer to a polymer of amino acid residues. The terms apply to naturally occurring amino acid polymers, to amino acid polymers in which one or more amino acid residue is an artificial chemical analogue of a corresponding naturally occurring amino acid, as well as to amino acid polymers having one or more tags or any other modification. Specific examples of proteins include antibodies, enzymes and some types of antigens.

The term “nucleic-acid molecule” as used herein also may refer to a nucleic-acid of known sequence or source, a nucleic-acid of interest or a nucleic-acid to be introduced into cells. As referred to herein, the terms “nucleic-acid”, “nucleic-acid molecules” “oligonucleotide”, “polynucleotide”, and “nucleotide” may interchangeably be used herein. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may include sense and antisense oligonucleotide or polynucleotide sequences of DNA or RNA. The DNA or RNA molecules may be, for example, but not limited to: complementary DNA (cDNA), genomic DNA, synthesized DNA, recombinant DNA, or a hybrid thereof or an RNA molecule such as, for example, mRNA, shRNA, siRNA, miRNA, Antisense RNA, and the like. Each possibility is a separate embodiment. The terms further include oligonucleotides composed of naturally occurring bases, sugars, and covalent inter-nucleoside linkages, as well as oligonucleotides having non-naturally occurring portions, which function similarly to respective naturally occurring portions. The term nucleic acid molecules encompass “nucleic acid construct” and “expression vector”. The terms nucleic acid construct” and “construct” may interchangeably be used. The terms refer to an artificially assembled or isolated nucleic-acid molecule which may include one or more nucleic-acid sequences, wherein the nucleic-acid sequences may include coding sequences (that is, sequence which encodes an end product), regulatory sequences, non-coding sequences, or any combination thereof. The term construct includes, for example, vector but should not be seen as being limited thereto. The term “Expression vector” refers to constructs that have the ability to incorporate and express heterologous nucleic-acid fragments (such as, for example, DNA), in a foreign cell. In other words, an expression vector comprises nucleic-acid sequences/fragments (such as DNA, mRNA, tRNA, rRNA), capable of being transcribed. Many prokaryotic and eukaryotic expression vectors are known and/or commercially available. Selection of appropriate expression vectors is within the knowledge of those having skill in the art. In some exemplary embodiments, the expression vector may encode for a double stranded RNA molecule in the target site. In some embodiments, the expression vector may encode for a marker in the cells, such that upon expression of the marker the cells may be visualized by imaging methods known in the art. In some embodiments, nucleic acid molecules may be provided as is or in a suitable solution/fluid/medium.

The term “expression”, as used herein, refers to the production of a desired end-product molecule in a target cell. The end-product molecule may include, for example an RNA molecule; a peptide or a protein; and the like; or combinations thereof. In some the expression may be identified by identifying the end product in the cell, for example, by biochemical methods, analytical methods, imaging methods, and the like.

As used herein, the terms “introducing” and “transfection” may interchangeably be used and refer to the transfer of molecules, such as, for example, nucleic-acids, polynucleotide molecules, vectors, and the like into a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein.

As used herein, the terms “substrate”, “slide”, and “cell slide” may interchangeably be used. The terms are directed to a solid or semi solid substrate onto which cells or sets of molecules may be seeded, deployed, dispensed, dispersed, attached, adhered, tethered, placed, grown, and the like. In some embodiments, cells carried by the substrate may be transfected or may be deposited with other molecules. In some embodiments, the substrate may have any regular or irregular shape, such as, rectangular, circular, elliptical, and the like. In some embodiments, the substrate may have a substantially flat planar surface. The substrate may be made of such materials as, glass, quartz, plastic, polystyrene, poly-propylene, synthetic or organic polymeric gels, and the like, or any combination thereof. Each possibility is a separate embodiment. In some embodiments, the substrate may be coated with various suitable materials, suitable for the cells or molecules deposited on the substrate. In some embodiments, the coating may be on the surface configured to bear the cells or molecules. In some embodiments, a suitable coating may include, for example, but not limited to: Poly-1-lysine, poly-D-lysine, Aminosilanes, Poly-1-ornithine, Collagen, Fibronectin, Laminin, or any combination thereof. Each possibility is a separate embodiment. In some embodiments, the coating may be on more than one surface of the substrate.

According to some embodiments, the substrate may be made of a solid, rigid or semi-rigid material designed to withstand stress and strain forces and/or withstand various temperatures. In some embodiments, the properties of the substrate are selected to match the assay in which it is used. In some embodiments, the substrate is transparent. In some embodiments, the substrate is opaque.

According to some embodiments, the substrate has a rectangular surface having a length in the range of about 2-30 cm. In some embodiments, the substrate has a rectangular surface having a length in the range of about 5-20 cm. In some embodiments, the substrate has a rectangular surface having a length in the range of about 7-15 cm. In some exemplary embodiments, the substrate has a rectangular surface having a length of approximately 7.5 cm. In some embodiments, the substrate has a width in the range of about 1-30 cm. In some embodiments, the substrate has a width in the range of about 5-20 cm. In some embodiments, the substrate has a width of approximately 2.5 cm. In some embodiments, the substrate has a depth in the range of about 0.01-1 cm. In some embodiments, the substrate has a depth in the range of about 0.05-0.5 cm. In some embodiments, the substrate has a depth of approximately 0.11 cm. According to some embodiments, the substrate has a rectangular surface having a length to width ratio in the range of about 1-10. According to some embodiments, the substrate has a rectangular surface having a length to width ratio in the range of about 2-5. According to some embodiments, the substrate has a rectangular surface having a length to width ratio of approximately 3. According to some embodiments, the substrate has a circular surface. In some embodiments, the substrate has a surface area of about 18.75 cm². In some embodiments, the substrate has a surface area in the range of about 1-500 cm².

As used herein, the terms “mesh” refer to a porous structure having multiple pores/apertures configured to allow controllable passage and/or retaining of liquid/cells/molecules through/within the pores/apertures. A mesh may be a film made of a network of wires, strands or threads, attached, woven or interlaced to form multiple apertures. According to some embodiments, the apertures of the mesh have a predetermined density and properties depending on the matter to be passed through and/or retained within the apertures, or according to the properties of the desired outcome/pattern. According to some embodiments, the mesh may have any desired pattern/structure. According to some embodiments, the mesh may be extruded, oriented, expanded, woven or tubular; the mesh may be made from connected or woven strands of polymer(s) (such as inert materials) that define a mesh structure with a mesh pattern confining the plurality of holes/apertures in the mesh. According to some embodiments, the mesh may have a weaving pattern confining the holes thereof. According to some embodiments, the mesh may have a lattice structure confining the holes thereof. According to some embodiments, a mesh may be a web, a net, a lattice, honeycomb pattern, matrix pattern, and the like. According to some embodiments, the mesh may be made of a polymeric material. In some embodiments, the mesh may be made from a hydrophobic material. In some embodiments, the mesh may be made from a polymeric hydrophobic material. In some embodiments, the mesh may be made from a material having low wettability. In some embodiments, the mesh may be made from a polymeric material having low wettability. In some embodiments, low wettability may be in the range of about 20-45 mN/m, and any subranges thereof. As referred to herein, the term “wettability” refers to the ability of a solid surface to reduce the surface tension of a liquid. The term “wetting” as used herein refers to the ability of a liquid to maintain contact with the solid surface.

According to some embodiments, the mesh and pores/apertures thereof are configured such that capillary forces are introduced when the mesh is approximated to or placed on a wet surface or when the mesh is wet before or after being placed on a surface. According to some embodiments, the mesh and pores/apertures thereof are configured such that capillary forces are introduced when the mesh is approximated to or is placed on a wet substrate or when the mesh is wet after or before being placed on the substrate.

According to some embodiments, a mesh may be configured to controllably avail/allow passage of viable cells or sets of molecules through the apertures thereof. A mesh may be termed herein “mesh”, “cell-mesh”, “cell-sheet”, “cell-seeding sheet”, “cell-seeding mesh”, “molecules mesh” and/or “seeding mesh”.

According to some embodiments, the properties of the mesh may be determined based on the material to be passed/transferred therethrought. In some embodiments, a mesh configured to controllably avail/allow passage of viable cells may be configured to have apertures having a size in the range of about 20-800 μm (micron). In some embodiments, the apertures may have a size in the range of about 50-500 μm. According to some exemplary embodiments, a cell-mesh may be configured to have apertures of approximately 100 μm in size. According to some embodiments, a cell-mesh may be configured to have apertures of any appropriate size. According to some embodiments, a mesh may have any density/concentration of apertures per area unit.

According to some embodiments, the mesh may be configured to controllably retain and/or avail/allow passage of molecules (such as, for example, nucleic-acid molecules, lipids, proteins, peptides, antibodies, antigens, beads, small organic molecules, and the like) and/or solutions containing such molecules, to a substrate. In some embodiments, the molecules are nucleic acid molecules. In some embodiments, the molecules are lipids. In some embodiments, the molecules are proteins or peptides. In some embodiments, the molecules are antibodies. In some embodiments, the molecules are antigens. In some embodiments, the molecules are beads. In some embodiments, the molecules are small organic molecules. According to some embodiments, a mesh configured for depositing sets of molecules may be configured to have apertures in the range of about 5-200 μm. According to some embodiments, a mesh configured for depositing sets of molecules may be configured to have apertures in the range of about 10-100 μm. In some exemplary embodiments, such molecules mesh may be configured to have apertures of approximately 41 μm. According to some embodiments, a molecules mesh is configured to have apertures of more than about 10 μm.

According to some embodiments, a mesh may be made of any suitable material. In some embodiments, the mesh may be made of a polymeric material or combination of such materials. In some embodiments, the mesh may be made from a polymeric material having low wettability (for example, in the range of about 20-45 mN/m). In some embodiments, the mesh may be made from a hydrophobic polymer. For example, a mesh may be made from such materials as, but not limited to: nylon, polyester, polyurethane, Polyethylene (PE) polyethylene terephthalate (PET), Polypropylene (PP), Polyvinyl chloride (PVC), Polytetrafluoroethylene (PTFE), Polyvinylidene fluoride (PVDF), Polydimethylsiloxane (PDMS), glass, and the like, or combinations thereof. Each possibility is a separate embodiment.

As used herein, the terms “restrainer”, “restraining grid”, “grid”, “cell restrainer”, and/or “molecules restrainer” refer to a material configured to be placed/mounted on, soaked, at least partially or completely within, or integrated in a mesh and to obstruct passage of liquids, cells, beads, molecules (such as nucleic-acid molecules, lipids, proteins or peptides, and the like) or solutions containing such molecules, in the mesh pores in which it is placed, soaked and/or integrated. According to some exemplary embodiments, the grid is configured to repel hydrophilic and/or water-based solutions, such as, for example, cell-containing solutions or molecules (such as nucleic-acid)-containing solutions from the regions in which it is placed, soaked and/or integrated. According to some embodiments, the grid is shaped to provide “restrainer-free” areas confined by the restrainer; the restrainer-free areas are configured to allow passage of cells, cell-containing solutions, beads, beads-containing solutions, molecules, and/or molecules-containing solutions. In some embodiments, the “restrainer-free” areas form a matrix/array.

According to some embodiments, the restrainer is shaped to form a network of lines that cross each other to form a series of squares or rectangles, or any desired form. According to some embodiments, the grid creates a matrix/array of regions/spots/small chambers/compartments/elements confined by the lines of the grid. In some embodiments, the grid may form an addressable array. According to some embodiments, the grid lines have predetermined thickness (line width and/or height) and spaced apart by a certain predetermined spacing according to the desired shape and area of the chambers/compartments/elements of the array. In some embodiments, the grid defines the perimeters of the chambers and/or the array.

According to some embodiments, the grid may be made of a liquid or semi-liquid hydrophobic material, capable of solidifying, which may be a thermoplastic polymer, thermally-cured, liquid-soluble polymer, a photo-initiated polymer, non-toxic hydrophobic material, and the like. In some embodiments, the grid is hydrophobic. In some embodiments, the grid is firm/stiff after solidifying. In some embodiments, the grid material is non-toxic to the cells.

According to some embodiments, the width (thickness) of the grid lines may be determined based on the cells/molecules to be transferred. According to some exemplary embodiments, a grid may be made of a non-toxic hydrophobic polymer arranged in perpendicular or semi-perpendicular lines having a width of about 0.3-5 mm and density of about 2-9 horizontal lines per cm (for example, about 2.9-5 horizontal lines per cm) and/or 2-9 vertical lines per cm (for example, 2.9-5 vertical lines per cm), resulting in molecules (such as, nucleic-acid molecules) hosting chambers with a surface area of in the range of about 0.9-9 mm², and any subranges thereof. Each possibility is a separate embodiment.

The terms “Array” and “matrix” as used herein refer to the arrangement of objects on a surface, so as to form an arrangement of separated chambers/compartments/elements. In some embodiments, the array is systematic. In some embodiments, the array may be formed by cross lines (for example, horizontal lines, vertical lines, diagonal lines, circular lines, and the like). In some embodiments, the array is arranged in the form of columns and rows. In some embodiments, the cross lines may be physical lines, or virtual lines, providing separation between the various elements/chambers/compartments of the array. In some embodiments, the array is an “addressable array” (also referred to as a “designated array”), that is, the location of the various chambers are identifiable and recognizable and each may be assigned an “address” which is indicative of its relative location within the array. In some embodiments, the shape, size, distribution and/or dimension of the compartments/chambers forming the array may be predetermined. In some embodiments, the form, shape, size, distribution and/or dimension of the array may be determined by the pattern of the grid on the mesh.

As used herein, the terms “transparency”, “transparency film”, “perforated transparency”, “transparency sheet”, “perforated sheet”, “perforated film” and/or “porous film” refer to a layer having multiple apertures configured to be placed on a cell-mesh. According to some embodiments, the transparency may be hydrophobic.

According to some embodiments, the apertures of the transparency are located at locations that match the chambers/spots of the cell-mesh on which it is configured to be placed. According to some embodiments, the apertures are located on the sheet at locations that are co-centric with center of each chamber/spot of the cell-mesh on which it is configured to be placed.

According to some embodiments, the apertures of the transparency are configured to allow controllable passage of viable cells there through for seeding a substrate. According to some embodiments, the diameter of the pores may range from 0.2 mm to 2 mm, or any suitable sub range thereof.

According to some embodiments, the transparency may be made of plastic, polyester, PET high density, PET low density, PP, PVC, polyurethane, PP, PVC, PTFE, PVDF, PE and/or other hydrophobic materials. Each possibility is a separate embodiment.

According to some embodiments, the transparency forces the cell-containing solution through the pores of the seeding mesh in a condensed drop. This contributes to the homogeneity of the deposited element in case relatively large drops are introduced to each chamber/spot on the cell-mesh and may eventually aid in maintaining a constant cell density on the slide.

As used herein, the terms “float” or “float device” refer to a device configured for removing a mesh (gridded or not gridded) or for facilitating the removal of a mesh from the substrate, subsequently to seeding cells or other molecules on the substrate, and/or subsequently to incubating the substrate with a mesh, without pilling cells off and/or without affecting the seeding/array pattern, either before or after it has been seeded with cells or molecules. The float is designed to carry a mesh, (optionally, with aligned transparency) and/or allow alignment of the mesh with the substrate or any other desired surface. In some embodiments, the float device is equipped with floating elements allowing detachment of the device from the substrate, at the end of the incubation period without pilling cells or molecules which have been attached to the substrate, from the substrate and/or without affecting the seeding/array pattern.

According to some embodiments, various interactions, such as, capillary forces allow interaction and attachment between the mesh and the substrate surface, forcing cells or molecules (e.g., nucleic-acid molecules) passing through the mesh to the substrate, to adopt/acquire the pattern of the mesh. Advantageously, the use of the mesh for the transfer of the cells or sets of molecules to the substrate provides a very efficient, accurate and cost effective manner to transfer the sets of molecules or cells to the substrate in a homogenous density pattern and in a predetermined array. According to some embodiments, provided are systems, kits, devices and methods for seeding cells or molecules on a substrate, using a seeding-mesh to deploy the cells or molecules onto the substrate. According to some embodiments, seeding cells or molecules to a surface of a suitable substrate using a seeding-mesh results in a seeding pattern determined by the pattern/characteristics/structure of the mesh. In some embodiments, the cells or molecules are constrained by the threads of the mesh and availed through the holes (apertures) of the seeding-mesh to be deposited in a desired pattern on the substrate. Advantageously, the use of the mesh for the cell-seeding results in a homogeneous controllable ordered seeding density across the surface of the substrate. In further embodiments, the use of such seeding mesh results in the cells being seeded in an array, which may advantageously be predetermined and/or addressable.

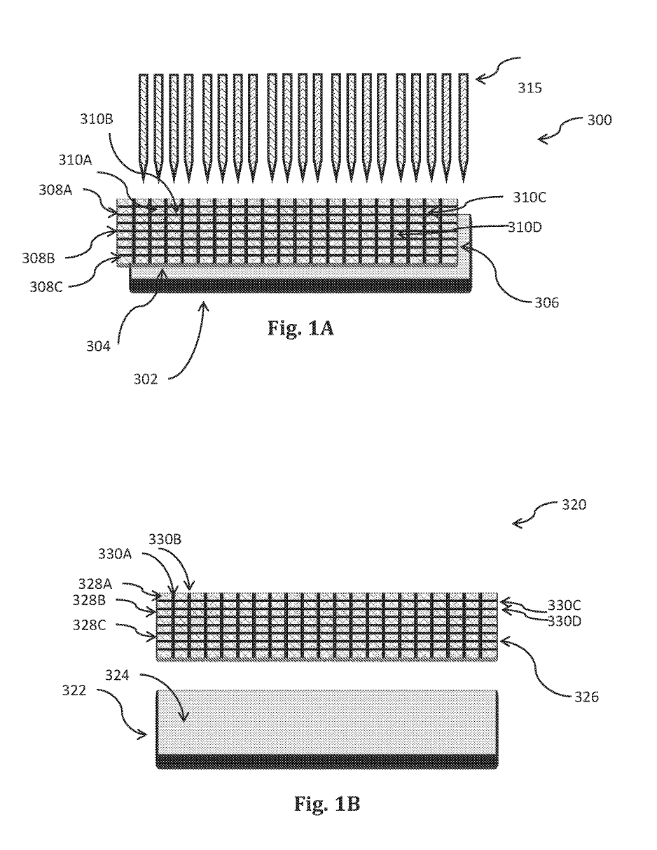

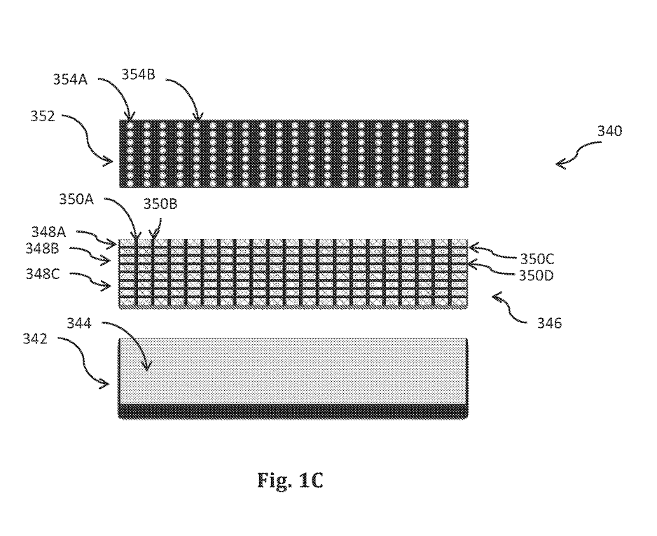

Reference is now made to FIG. 1A, which schematically illustrates a system for seeding cells or sets of molecules on a substrate, according to some embodiments. System 300 includes a substrate 302 having a surface 304 capable of attaching cells or other sets of molecules (such as, nucleic acids, lipids, proteins, peptides, antibodies, antigens, beads, small organic molecules, and the like), a mesh 306 having chambers (shown as exemplary representative chambers 310A-D) confined by a grid lines (shown as representative grid lines 308A-C). Further shown is dispenser 315, configured to dispense the cells/molecules (as is or in a suitable solution) on the mesh. According to some embodiments, dispenser 315 is configured to controllably deploy a plurality of predetermined cells or molecule types, optionally within a fluid. According to some embodiments, dispenser 315 may be configured to deploy varying volumes of cells or molecules. In some embodiments, dispenser 315 may have one or more nozzles configured to dispense cells or molecules. Mesh 306 is configured to be placed on substrate surface 304 and eventually confer the pattern thereof to the cells/molecules passing through it, as they are seeded/transferred to the substrate. Grid 310 is configured to obstruct passage of the cells or molecules in predetermined areas, thereby to form/confine chambers, arranged in an array. According to some embodiments, capillary forces are generated upon placing mesh 306 on surface 304, resulting in fastening mesh 306 to surface 304 and transfer of the cells or molecules through the mesh to the substrate, in accordance with the pattern of the mesh.

Reference is now made to FIG. 1B, which schematically illustrates a kit for seeding cells or sets of molecules on a substrate, according to some embodiments. Kit 320 includes a substrate 322 having a surface 324 capable of attaching cells, beads or other sets of molecules (such as, nucleic acids, lipids, proteins, peptides, antibodies, antigens, small organic molecules, and the like), a mesh 326 having seeding chambers (shown as exemplary representative chambers 328A-C) confined by grid lines (shown as representative grid lines 332A-D). The kit may further include a frame, configured to allow maintaining the orientation/alignment of mesh 326 and surface 324, to eventually form an array of cells or sets of molecules on the substrate surface.

Reference is now made to FIG. 1C, which schematically illustrates an exemplary cell-seeding kit 340, according to some embodiments. Cell-seeding kit 340 includes a substrate 342 with a surface 344 suitable for adherence or attachment of viable cells, a seeding mesh 346 having seeding chambers 348A-C confined by grid lines (shown as representative grid lines 350A-D) and an optional perforated film 352 having a plurality of apertures (shown as exemplary apertures 354A-B). Apertures (such as apertures 354A-B) are configured to avail passage of viable cells therein for being deployed onto mesh 346 in seeding spots 354. According to some embodiments, each one of apertures 354 is located on a predetermined location on film 352 such that upon approximating film 352 to mesh 346, apertures 354 match seeding chambers 348.

Mesh 346 is configured to be placed on surface 344. Grid 350 is configured to obstruct passage of viable cells in predetermined areas, thereby to confine seeding chambers 348. According to some embodiments, capillary forces are generated upon placing mesh 346 on surface 344 in the presence of fluid, resulting in fastening mesh 346 to surface 344. According to some embodiments, capillary forces are generated upon placing film 352 on mesh 346 in the presence of fluid, resulting in fastening film 352 to mesh 346.

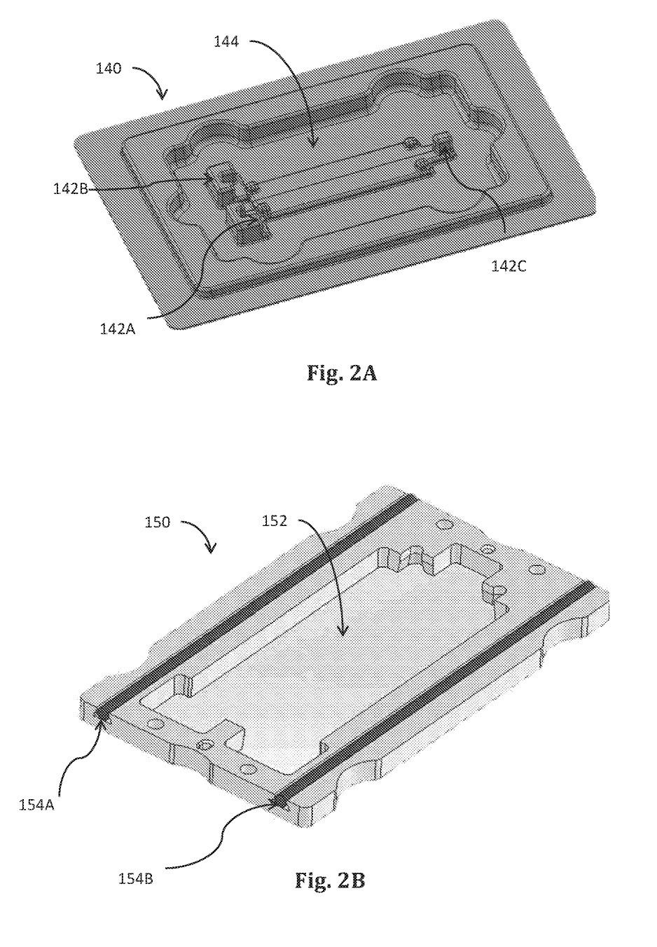

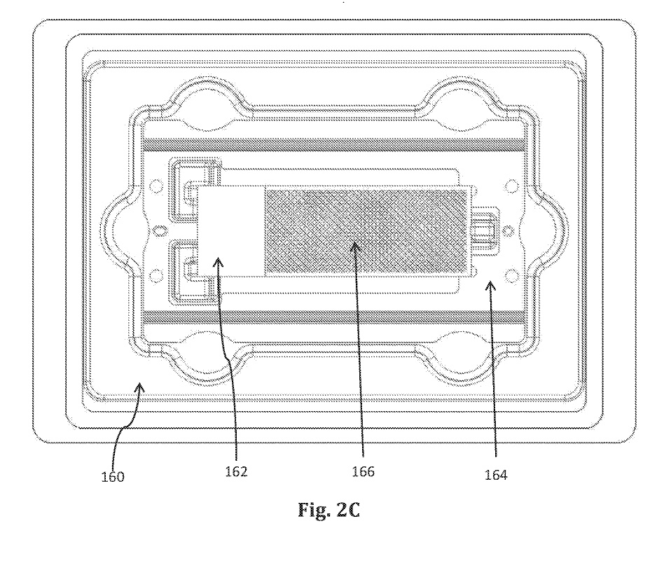

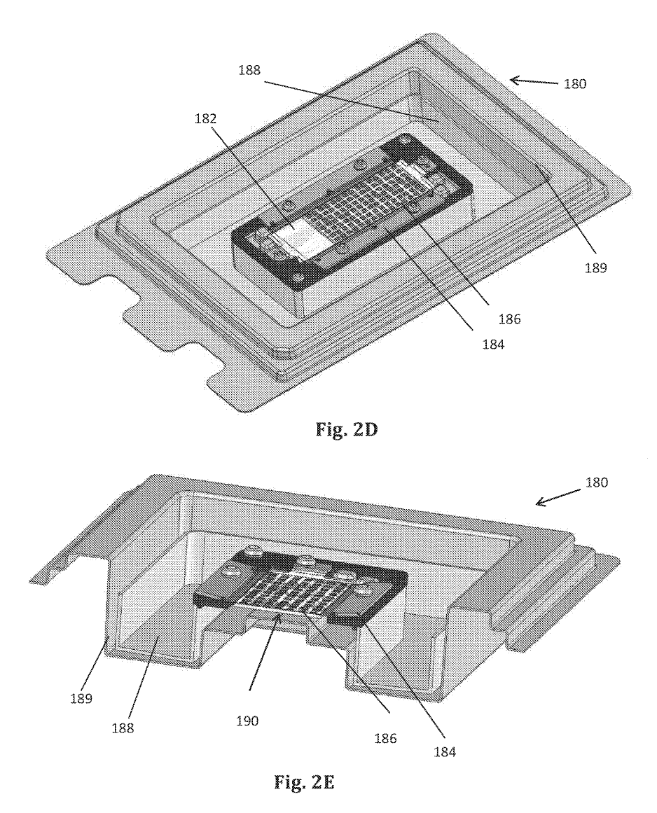

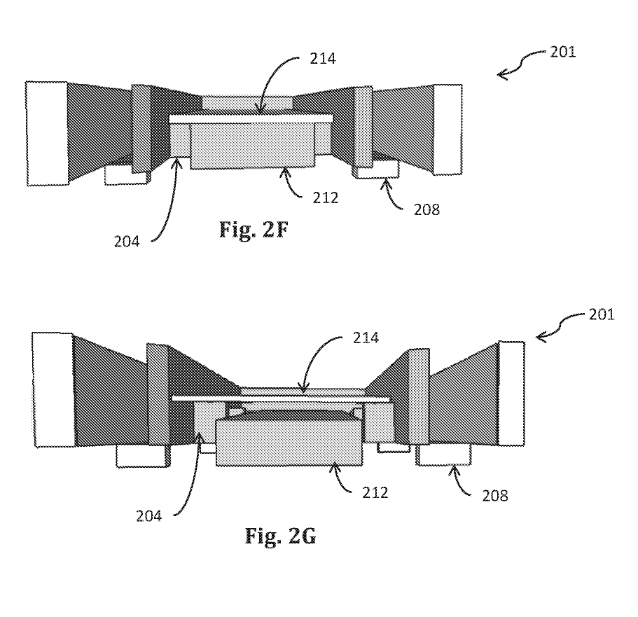

Reference is now made to FIG. 2A, which illustrates a perspective view of a substrate casing configured to hold a substrate having a surface suitable for attachment of cells or sets of molecules. As shown in FIG. 2A, substrate casing, 140 includes clamping (or mounting) elements (shown as elements 142A-C), configured to hold and secure the substrate (for example, a slide) to its location. Casing 140 forms a shallow region/space, 144, which allows drainage of excess fluid (such as cell medium or any other suitable medium) during the flooding process. The walls of the casing, (unlabeled) may be higher than the substrate surface when it is positioned in its position (groove), such that the substrate top surface is immersed in fluid (e.g. growth medium) during incubation periods.

Reference is now made to FIG. 2B, which illustrates a top perspective view of a mesh-holding frame configured to hold and stretch a mesh for further manipulation. Mesh frame, 150, in FIG. 2B is shown in the form of a rectangular frame, having an internal open space 152 over which the mesh may be placed/positioned/stretched. Mesh frame 150 may further include sealing/stretching/fastening elements (shown as elements 154A-B), configured to provide uniform stretching of the mesh and to secure it in place.

In some embodiments, the substrate casing and the mesh frame may have similar or matching dimension, so as to allow alignment and fitting of the mesh frame and the substrate casing, such that when the two are approximated, the mesh, secured in the mesh frame may be aligned to the substrate held in the substrate casing, to result in alignment of molecule sets deposited on the mesh or with cell groups attached to the substrate.

Reference is now made to FIG. 2C, which illustrates a perspective top view of a substrate casing (holding a substrate) and a mesh-holding frame (holding a mesh), the mesh frame being positioned on top of the substrate casing. As shown in FIG. 2C, substrate casing 160, holds substrate 162 (shown in the form of a slide). Further shown is mesh-holding frame 164, positioned on the substrate casing, such that mesh 166 (shown as gridded mesh) is aligned/positioned over the substrate. The alignment/positioning of the substrate casing and the mesh-holding frame may be achieved by various means, such as, for example, but not limited to, visual means (for example, corresponding markers on each of the casing and frame), physical means (for example, matching grooves and protrusion, assuring alignment and correct positioning of the frame and casing), and the like. Upon positioning of the mesh over the substrate, the cells or molecules may be deposited on the mesh and transferred through the mesh to the substrate, spontaneously or upon further manipulation, such as, for example, addition of a fluid. In some embodiments, the mesh and the substrate may be incubated for any desired length of time, within the casing. In some embodiments, the incubation is performed in the presence of a suitable fluid (such as, for example, but not limited to: cell medium, buffers, solutions, isotonic-solutions, preservative-solutions, reagent mixes, and the like or any combinations thereof). In some embodiments, the casing may further be used during separation of the mesh from the substrate in the presence of additional fluid, either with or without providing a float mechanism allowing such separation, without affecting the cells. In some embodiments, the float may be attached to the mesh frame prior to being is placed on the substrate. In some embodiments, the casing may further be used for incubation of the substrate with or without the mesh, immersed or not immersed in fluid/solution (such as for example, growth medium).

According to some embodiments, after the seeding/transferring process is completed, the mesh (and the mesh frame) may be detached/separated from the substrate, without affecting the cells (or molecules deposited), i.e. without pilling, compromising the seeding pattern and/or otherwise harming the cells or molecules on the substrate.

Reference is now made to FIGS. 2D-E which schematically illustrate the float device in the substrate casing, which is configured to facilitate separation between the mesh and the substrate, without harming the cells or molecules deposited on the substrate, according to some embodiments. FIG. 2D illustrates a perspective top view of a framed casing (180) holding a substrate (182) and a mesh-holding frame (184), holding a mesh (186), the mesh frame being positioned on top of the substrate casing and optionally being attached, connected to or associated with a flotation means (device) (188, shown as a rectangular floatation device). In some embodiments, the flotation means may be attached to the mesh frame permanently or transiently. In some embodiments, the flotation means may be an integral part of the mesh frame. In some embodiments, the flotation means may be placed in the substrate framed casing. In some embodiments, the float device while attached to the mesh frame may be placed at the bottom of trench of the substrate framed casing during mesh-slide incubation in the absence of liquid. Reference is now made to FIG. 2E, which schematically illustrates a cross section view of the substrate casing (180), holding a mesh-holding frame (184), holding a mesh (186), the mesh frame optionally being attached, connected to or associated with a flotation means/device (188), during incubation with the slide and subsequently, as fluid is added to the interface (190) between the mesh and the substrate by dripping the fluid on top the mesh. Addition of the fluid to the mesh-slide interface may cancel out capillary forces between the two and provide separation of the mesh from the substrate. In some embodiments, as fluid is added, the mesh may further separate and distant from the substrate. In some embodiments, the mesh may float away from the substrate. In some embodiments, excess liquid may be drained to trench (189), which results in separation of the mesh from the substrate, as facilitated by the flotation device (188) that lifts the mesh frame from the substrate, as excess fluid accumulates in the trench (189), lifting the float up. The separation of the mesh from the substrate is achieved without harming or otherwise affecting the cells or molecules deposited on the substrate through the mesh.

FIG. 2F schematically illustrates yet another float-device in a cross section view, according to some embodiments. Shown is float-device 201 hosting mesh 214 and substrate 212, respectively. The framed container holding the substrate and mesh is not shown. Capillary forces fasten mesh 214 to slide 212 when the float-device 201 is placed over the substrate, aligning the mesh 214 over substrate 212. According to some embodiments, spacers 208 are designed to prevent the float from dropping down all the way to the bottom of the substrate casing trench in the absence of liquid, thus creating too much pressure on the slide-mesh interface. Further shown are protruded rods, 204, designed to obstruct passage of the mesh. FIG. 2G schematically illustrates a cross section of a float device 201 hosting substrate 212, separated from mesh 214, according to some embodiments. Throughout the separation, mesh 214 is carried by rods 204 as float rises up. According to some embodiments, separation between mesh 214 and substrate 212 is done by introducing a fluid configured to cancel out the capillary forces between mesh 214 and slide 212 thereby unfasten the connection between them as well as causing the float device 201 to rise up thus, lifting mesh 214 away from slide 212.

In some embodiments, the substrate casing and/or the mesh-holding frame may be made of any suitable material. In some embodiments, the substrate casing and/or the mesh-holding frame may be made of serializable material. According to some embodiments, the frame and/or casing are to withstand sterilizing procedures, such as, for example, an autoclave, chemiclav, gamma radiation, chemical sterilization, gas sterilization, a dry heat sterilizer, and the like.

According to some embodiments, the fluid introduced for seeding on the substrate and/or for the separation between the substrate and the mesh may be any water-based solution, such as an isotonic solution. For example, various appropriate cell culture media or buffers, such as, PBS, TBS, and the like may be used.

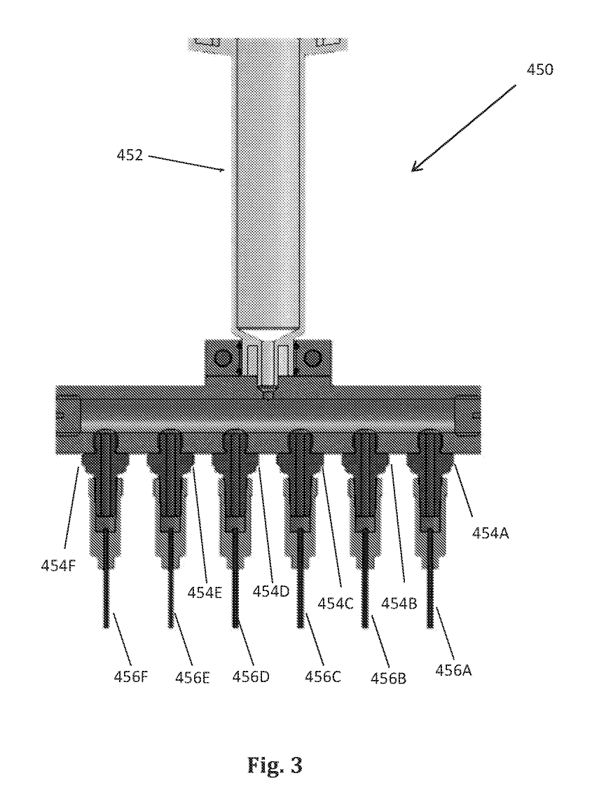

Reference is now made to FIG. 3, which illustrates a perspective front view of an exemplary dispenser, according to some embodiments. Exemplary dispenser 450 includes a cartridge reservoir 452, configured to allow maneuvering/operation of the dispenser and to optionally further hold cells or molecules (for example in a solution) to be dispensed on the substrate. Dispenser 450 further includes one or more separable printing tips/nozzles (shown as printing tips 454A-F). In some embodiments, the tips may be permanent or disposable. In some embodiments, the tips may have disposable, changing or replaceable ends, configured to be reversibly situated on the end of the tip. Shown in FIG. 3 exemplary disposable tip ends 456A-F, situated on the respective tips, 454A-F. In some embodiments, the printing tips may be identical or different in structure, composition and operation. In some embodiments, the tips may operate simultaneously or sequentially in a different, similar or identical manner. Each tip may dispense the same type of cells or molecules or different types of cells or molecules, depending on the setting of the dispenser and if/what type of reservoir is used. In some embodiments, each tip may dispense an equal amount/volume of cells or molecules. In some embodiments, each tip may dispense a different amount/volume of cells or molecules. In some embodiments, the dispenser tip(s) are positioned so as to align with matching chambers (situated in an array on the mesh), such that the type and/or composition and/or the amount/concentration of the cells or molecules dispensed to each chamber is known and addressable.

According to some embodiments, there is provided a substrate having a cell-carrying surface configured to carry viable cells; the substrate may have a planar, flat or semi-flat surface configured to carry, hold, attach viable cells. According to some embodiments, there is provided a substrate having a cell-carrying surface configured to carry viable cells; the substrate may have a planar, flat or semi-flat surface configured to carry, hold, attach viable cells.

According to some embodiments, there is provided a substrate (for example, a slide) having a cell carrying surface configured to carry, attach, bear, viable cells; and a plurality of viable cell groups located at predetermined distinct locations on the cell carrying surface, wherein the cell carrying surface comprises cell-free space configured to provide spatial separation between the cell groups.

According to some embodiments, there is provided a substrate having a cell-carrying surface configured to carry viable cells; the substrate may have a planar, flat or semi-flat surface configured to carry, hold, attach viable cells; a plurality of viable cell groups disjointedly seeded at known distinct locations on the surface, wherein on the surface there are cell-free areas providing spatial separation between different cell groups.

In some embodiments, the mesh is configured to develop capillary forces with the surface of the substrate. In some embodiments, the mesh is configured to develop capillary forces with the surface of the substrate.

In some embodiments, the cell-free or molecule-free space on the substrate is formed by the hydrophobic material soaked within or deposited on the mesh, the hydrophobic material is configured to repel/repulse cells or molecules (or water based solutions containing the same), to thereby form free space on the substrate while forming chambers confined by the free space.

According to some exemplary embodiments, the hydrophobic polymer is arranged on the mesh in horizontal and vertical lines forming a hydrophobic polymer matrix such that the chambers are rectangular spots/chambers confined or bordered by the horizontal and vertical lines.

According to some embodiments, the hydrophobic polymer on the mesh forms a grid such that the chambers are confined in a matrix/array. In some embodiments the array may be an addressable array, a predetermined array, and/or a designated array.

According to some embodiments, the cell-free space or molecule-free space on the mesh comprises cell/molecules-free horizontal and vertical lines forming a cell-free or molecule-free grid such that cell groups or sets of molecules are arranged in rectangular shapes confined or bordered by the cell-free horizontal and vertical lines.

According to some embodiments, the resulting cell groups or sets of molecules are positioned/located on the substrate surface in a matrix pattern with a cell-free or molecule-free space providing/allowing separation between the groups, to form an array. In some embodiments, the array may be an addressable array, a predetermined array, and/or a designated array.

According to some embodiments, the cells or molecules are homogeneously deposited on the surface of the substrate. According to some embodiments, the density of cells or molecules deposited on the surface of the substrate is homogeneous.

According to some embodiments, the cells are deposited on the substrate surface in multiple disjoint cell groups at different locations on said substrate with homogeneity in cell distribution between said groups. According to some embodiments, the molecules are deposited on the substrate surface in multiple disjoint sets of molecules at different locations on said substrate with homogeneity in molecules distribution between said sets.

According to some embodiments, there is provided a cell seeding device, having: a substrate having a seeding surface configured to facilitate attachment/carry cells, and a seeding mesh having a patterned structure, configured to avail passage of viable cells to said surface, wherein the deploying of the viable cells is controlled/restricted by the structure/pattern of said mesh.

According to some embodiments, the mesh may be extruded, oriented, expanded, woven or tubular; the mesh may be made from connected strands of polymers or other inert materials that defines a mesh structure with a mesh pattern confining the plurality of holes/apertures in the mesh. According to some embodiments, the mesh may have a weaving pattern confining the holes thereof. According to some embodiments, the mesh may have a lattice structure confining the holes thereof. According to some embodiments, a mesh may be a web, a net, a lattice and the like.

According to some embodiments, there is provided a cell seeding device or kit that may include a cell slide which may have a seeding surface configured to carry viable cells; and a cell seeding mesh. The seeding mesh is configured to avail/allow passage of the viable cells to the seeding surface or any portion thereof; a cell restrainer on or within the seeding mesh, placed, printed or deposited in predetermined areas and configured to obstruct passage of cells, thereby creating seeding chambers on the seeding sheet, through which passage of cells is permitted and other spaces within the seeding mesh through which passage of cells is blocked/restrained; wherein the seeding mesh is configured to be placed on the seeding surface, the latter being configured to receive cells deployed via the seeding mesh, thereby creating cell groups on the seeding surface and cell-free area that provides spatial separation between the cell-groups.

According to some embodiments, the cell seeding device may further include a detacher (lifting element) configured to detach (to lift) the seeding mesh from the seeding surface.

According to some embodiments, the device or kit may further include a perforated layer configured to be mounted on said cell seeding mesh, said perforated layer comprises a plurality of apertures configured to allow passage of viable cells there through to said seeding mesh.

According to some embodiments, the cell seeding device or system may further include an incubator configured to provide predetermined conditions to the surrounding environment of the cells/molecules on the substrate.

According to some embodiments, the cell seeding mesh may be a 20-800 μm polymeric mesh.

According to some embodiments, the cell restrainer comprises a hydrophobic polymer absorbed within the mesh. The hydrophobic polymer is configured to repel/repulse cells (and/or cell-containing solution), thereby providing a cell-free space within the mesh and cell transfer chambers confined/bordered by the hydrophobic polymer.

According to some embodiments the hydrophobic polymer is arranged in a grid structure comprising horizontal lines and vertical lines, such that the seeding chambers are rectangular spots located in distinct predetermined locations and confined by the horizontal and vertical lines. In some embodiments, the grid may be made of a liquid or semi-liquid hydrophobic material, capable of solidifying. In some embodiments, hydrophobic polymer may be a thermoplastic polymer, thermally cured, liquid soluble polymer, a photo-initiated polymer, non-toxic hydrophobic material, and the like. In some embodiments, the grid is firm/stiff after solidifying. In some embodiments, the grid material is non-toxic to the cells.

According to some embodiments, the cell seeding device may include a detacher configured to detach the seeding mesh, from the seeding surface, essentially without affecting the cells on the surface. In some embodiments, fluid may be used to facilitate detaching of the seeding mesh from the substrate. According to some exemplary embodiments, after the incubation period of the mesh and substrate is complete, a, fluid/solution (for example, a growth medium) may be added to the mesh-substrate interface (for example, by dripping fluid on top the mesh while mounted on the substrate), thus eliminating the capillary forces between the two elements as well as facilitating floating of the mesh. This allows removal of the mesh without pilling of newly deposited cells/molecules off the substrate.

According to some embodiments, the cell seeding device, may further include an incubator configured to provide predetermined conditions to a surrounding environment of the cells/molecules on the substrate.

According to some embodiments, there is provided a cell dispensing system, that may include a substrate configured to carry/attach cells; a mesh configured to avail/allow passage of the cells to the seeding surface or any portion thereof; the cells acquire the mesh pattern when deposited on the substrate; a restrainer at least partially absorbed or deposited on the mesh at a predetermined space, the restrainer is configured to repel/repulse cells, thereby providing cell-free space within the mesh and cell chambers confined by the restrainer; and a cell dispenser (printer) configured to deploy cells to the chambers.

According to some embodiments, there is provided a device for depositing sets of molecules on a substrate, the device may include substrate which may have a surface configured to accept the sets of molecules; and a mesh. The mesh is configured to avail/allow passage of the sets of molecules to the surface or any portion thereof, the sets of molecules acquire the mesh pattern when deposited on the substrate; a restrainer on or within the mesh, printed, deposited or placed in predetermined areas and configured to obstruct passage of molecules, thereby creating receiving chambers on the seeding sheet, through which passage of molecules is permitted and other spaces within the seeding mesh through which passage of the molecules is blocked/restrained; wherein the deploying mesh is configured to be placed on the receiving surface, the latter being configured to receive the sets of molecules deployed via the mesh, thereby creating sets of molecules regions on the surface and molecules-free area that provides spatial separation between the sets of molecules, such that the sets of molecules are arranged in an array on the substrate. In some embodiments, the molecules may be selected from, but not limited to: nucleic acids, proteins, peptides, antibodies, enzymes, antigens, lipids, small organic molecules, beads and the like or any combination thereof. In some embodiments, the surface may be coated with a suitable layer which allows the attachment/receiving of the respective molecules on the surface of the substrate.

In some embodiments, there is provided an array of molecules such as proteins, peptides, antibodies, enzymes, lipids, nucleic acids, polymers, metals or antigens, deposited on a suitable substrate, via a mesh, to result in homogenous patterning of the molecules on the substrate, wherein the dispersion pattern of the molecules is determined by the pattern of the mesh. Such an array may be used for further downstream applications, including biochemical methods, imaging methods and/or molecular biology methods

In some embodiments, there is provided an array of beads homogenously deposited on a substrate capable of attaching thereto. In some embodiments, binding or attachment of the beads to the substrate may be covalently, electrically, avidin-biotin interaction, or otherwise. In some embodiments, the beads can be further linked to any type of molecule, including, for example, biologically active molecules (such as antibodies, enzymes, and the like), biological substrates (such as cell lysates and the like), chemically reactive molecules (such as chelators, indicators and the like), electrically charged molecules, and the like, or combinations thereof. Different beads may be deployed in different chambers (i.e. sets of beads). By utilizing the systems, devices and methods disclosed herein, a cost effective and highly efficient means of generating highly ordered arrays of large particles (such as beads) are provided. Additionally or alternatively, large surfaces of particles (such as magnetic particles) highly ordered in a desired pattern on the substrate may be formed and used as templates for different applications such as precipitation, conducting electric currents etc.

According to some embodiments, there is provided a method for seeding cells on a substrate having a surface configured to carry cells, the method may include one or more of the steps of:
a. Providing a mesh having a desired pattern, optionally comprising a cell restraining grid structure;
b. Placing the seeding sheet on the surface of a substrate, having a surface onto which cells may be attached;
c. Deploying, dispensing, aliquoting, cells to the substrate through the mesh, thereby obtaining a plurality of viable cell groups seeded on the surface of the substrate according to the pattern of the mesh and optionally according to the pattern dictated by the cell restraining grid structure (i.e., in an array); and
d. Separating/removing/detaching the mesh from the substrate, without harming, damaging and/or peeling the cells attached to the substrate, while maintaining the spatial separation of the cells within the array on the substrate.

According to some embodiments, the method of seeding may further include in step b) i) Providing a perforated hydrophobic layer (film) which may have multiple apertures located at different locations on the layer matching with the cell restraining grid structure; and ii) Placing the perforated hydrophobic layer on the mesh.

According to some embodiments, the seeding method may further include incubating the substrate before, during and/or after the separation from the mesh.

According to some embodiments, there is provided a method for seeding cells on a seeding substrate, the method comprising:
a. Providing a cell seeding mesh having a patterned structure;
b. Mounting the seeding mesh on a surface of a seeding substrate; and
c. Dispensing cells in a physiologically acceptable solution to the surface of the seeding substrate through the mesh, thereby obtaining multiple cells attached to the surface of the substrate according to the pattern of the mesh;

In some embodiments, the method may further include providing a fluid layer between the surface of the substrate and the mesh to facilitate the release of the viable cells from the mesh to the substrate, in a pattern of the mesh. In some embodiments, the method may further include providing a fluid layer between the surface of the substrate and the mesh to facilitate attachment between the substrate and mesh such that the viable cells passing from the mesh to the substrate, acquire the pattern of the mesh. In some embodiments, the pattern is a weave pattern.

In some embodiments, the method may further include separating the mesh from the surface of the substrate without compromising the seeding pattern and/or the vitality of the cells. In some embodiments, the separation may be facilitated/achieved by adding fluid to the mesh-substrate interface.

In some embodiments, the mesh comprises a grid structured such that upon dispensing the viable cells to the substrate through the mesh, a plurality of viable cell groups are obtained on the surface of the substrate, such that the cell groups are deposited in an array, determined by the grid on said mesh.

In some embodiments, the cells are viable cells. In some embodiments, there method may further include a step of incubating the cells before, during or after mounting the mesh on the surface of the substrate.

According to some embodiments, there is provided a method for depositing multiple sets of molecules in a predetermined array on a surface of a substrate, the method comprising:
a) Providing a mesh having a patterned structure;
b) Mounting the mesh on the surface of the substrate; and dispensing molecules in an acceptable solution (suitable medium) to the surface of the substrate through the mesh, thereby obtaining multiple sets of molecules in a predetermined array on the surface of the substrate.

In some embodiments, a controlled deposition density of the molecules is obtained.

In some embodiments, the method may further include providing a fluid layer between the surface of the substrate and the mesh to facilitate transfer/release of the molecules from the mesh to the substrate. In some embodiments, the method may further include providing a fluid layer between the surface of the substrate and the mesh to facilitate attachment between the substrate and mesh to thereby facilitate release of the molecules from the mesh to the substrate. In some embodiments, providing the fluid layer between the surface of the substrate and the mesh facilitates forcing the molecules to acquire the mesh pattern while the molecules pass through the mesh to the substrate.

In some embodiments, the method may further include separating the mesh from the surface of the substrate without compromising the deposition pattern of the molecules on the substrate. In some embodiments, separating the mesh from the surface of the substrate is facilitated by adding fluid to the mesh-substrate interface.

In some embodiments, the method may further include a step of providing the surface of the substrate with a coating layer conducive to the attachment of the sets of molecules. In some embodiments, the coating layer is homogeneously coated, deposited on or formed with said surface.

In some embodiments, the mesh comprises a grid structured such that upon dispensing the molecules to the substrate through the mesh, a plurality of sets of molecules are obtained on the surface of the substrate, such that the sets of molecules are deposited in an array, determined by the grid on said mesh.

In some embodiments, the molecules are nucleic acid molecules. In some embodiments, the molecules are proteins. In some embodiments, the molecules are peptides. In some embodiments, the molecules are antibodies. In some embodiments, the molecules are enzymes. In some embodiments, the molecules are metals. In some embodiments, the molecules are biological fluids or extracts. In some embodiments, the molecules are lipids. In some embodiments, the molecules are beads. In some embodiments, the molecules are small organic molecules. In some embodiments, the method may further include a step of incubating the molecules before, during or after mounting the mesh on the surface of the substrate.

According to some embodiments, there is provided a method for depositing sets of molecules on a substrate having a surface configured to receive the molecules, the method may include one or more of the steps of:
a. Providing a mesh having a desired pattern, optionally comprising a molecule restraining grid structure;
b. Placing the mesh on the surface of the substrate, which may be optionally coated with a coating layer;
c. Deploying, dispensing, aliquoteing, the molecules to the substrate through the mesh, thereby obtaining a plurality of sets of molecules deposited on the surface of the substrate according to the pattern of the mesh and optionally constrained/bordered by the molecules-restraining grid structure (i.e., in an array); and
d. Separating the mesh from the substrate, without harming, damaging and/or peeling the molecules attached to the substrate, while maintaining the spatial separation of the molecules within the array on the substrate.

According to some embodiments, the seeding method may further include incubating the substrate before, during and/or after the separation from the mesh.

According to some embodiments, the systems and devices disclosed herein may utilize one or more automatic or semi-automatic means/applicators. For example, depositing/dispensing/printing of cells, sets of molecules and/or grids may be performed by such automated or semi-automated dispensers, printers and/or applicators, each capable of applying a desired amount/concentration/volume of a desired cell, molecule or substance at a desired location in a an accurate manner.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

### EXAMPLES

### Example 1: Cell Seeding

Exemplified herein is a cell seeding method for achieving inter-slide and intra-slide homogeneity of cell seeding by using a nylon mesh at the cell-slide (substrate) interface.

According to the example, seeding is performed using a 100 μm nylon mesh (Merk Millipore, cat no. NY1H00010) stretched over a dedicated mesh-frame. The mesh is printed with vertical and horizontal liquid, polymeric, hydrophobic, thermoplastic material (PVC-based) lines, which are non-toxic to cells, to form an array of chambers (seeding spaces/spots) each confined by the vertical and horizontal hydrophobic lines. For example, the thickness of the printed lines on the mesh may have a 1.5 mm thickness and 2 mm×2 mm chambers (seeding spaces/spots) generating square chambers of about 3.5 mm pitch. The grid may solidify on the mesh by exposure to heat through baking for 20 min in an oven pre-heated to 100° C.

A Poly-L-Lysine coated slide (Polysciences cat no. 22247) is positioned in a substrate carrying case.

Then, mesh-holding frame carrying the nylon mesh is placed/aligned within the substrate carrying case, such that it is exactly aligned with the designated contours of the substrate carrying case and hence aligned with the substrate.

After the substrate and mesh aligned; 100-400 μl of full medium may be dispensed over the upper side of the mesh, or at a later stage, as detailed below. The medium may be any suitable medium, depending on the type of cells and downstream assay. In one example, the medium is MEM eagle Earle's salts base supplemented with 10% FBS, 1× Pen-strep solution, 1 mM Sodium Pyrovate and 2 mM L-glutamine; (Biological Industries, cat no. 01-040-1A, cat no. 04-127-1A cat no. 03-031-1B cat no. 03-042-1B, cat no. 03-020-1B, respectively).

Next, cell suspension is dispensed. In one example, Hela cells are seeded at about 1*10⁴ cells/μl (=10⁶ cells/ml) suspension in full medium by automated means; ˜350 nl/chamber. The cell suspension is dispensed to the center of each chamber in the array.

After the seeding step, the mesh and substrate are incubated in the substrate casing, with the lid on, for 30 minutes at 37° C.

Then, 2-3 ml of full medium is dripped on the top of the mesh such that the slide-mesh interface is flooded allowing the mesh to float above the slide such that it may be removed without pilling off cells. Then, incubation at 37° C. is carried until use in downstream assays.

### Experimental Protocol:

1. Stretch a 100 μm nylon mesh (Merk Millipore, cat no. NY1H00010) on a mesh-holding frame
2. Grid polymer squares (1.5 mm line width using 18 mm/sec dispenser motion speed; spacing of 2 mm×2 mm using a pitch/offset of 3.5 mm; 6×13 chambers according to the dimensions of the clear area on the slide) on a 100 μm nylon mesh with liquid, non-toxic hydrophobic thermoplastic material to generate clear chambers of 3.5 mm² and line thickness of 1.5 mm.
3. Solidify polymer grid by baking the meshes for 20 mins in an oven pre-heated to 100° C.
4. Place a Poly-L-Lysine coated slide (Polysciences cat no. 22247) in the slide casing.
5. Place the mesh-holding frame such that it is exactly aligned with the contour of the coated slide.

Drip 100 μl of full medium (MEM eagle Earle's salts base supplemented with 10% FBS, 1× Pen-strep solution, 1 mM Sodium Pyrovate and 2 mM L-glutamine; Biological Industries, cat no. 01-040-1A, cat no. 04-127-1A cat no. 03-031-1B cat no. 03-042-1B, cat no. 03-020-1B, respectively). Wait for the liquid to spread through the mesh.

5. Optionally, place on top the mesh a sheet of perforated transparency with holes aligned to the center of each chamber of the mesh (in this example, about 3.5 mm spacing). Remove any air bubbles. After the slide, mesh and transparency are perfectly aligned, add another 100 μl of full medium to the edges of the transparency such that it is better attached to the mesh. Wait for the liquid to spread.

6. Through the holes of the transparency or above the center of each mesh chamber, seed Hela cells at 1*10⁴ cells/μl suspension in full medium; 0.35 μl drop per hole. (automatically dispensing at: 0.05 sec/spot, 1 Bar air pressure, 1 cc syringe, 0.16 mm inner diameter needle)

7. Incubate cell slide with the mesh in an incubator at 37° C. for 30 minutes.

8. Flood floating device/mesh-slide interface with full medium such that capillary forces between the mesh and slide are eliminated. Gently remove the floating mesh (and transparency).

9. Incubate at 37° C. until use—preferably, an overnight incubation.

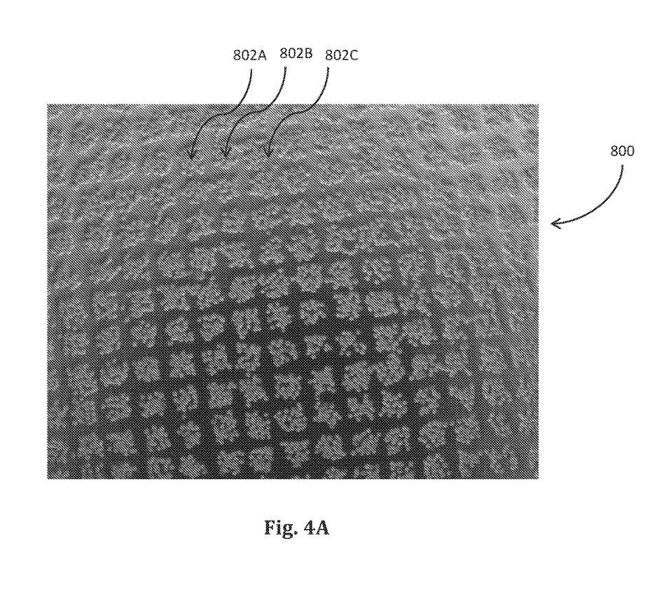

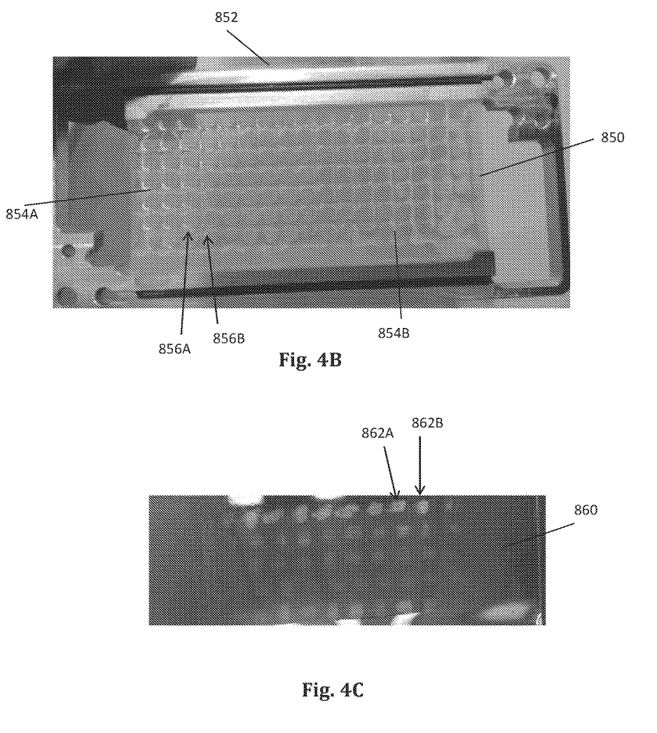

The results of the seeding performed as described herein are presented in FIG. 4A, which shows a pictogram of zoomed-in part of a cell slide (800), which carries multiple cells (shown as exemplary cells 802A-C), seeded at high degree of order and uniform density according to the pattern of the seeding mesh (weaving pattern of a 100 μm nylon mesh in this example). This represents the type of homogeneous seeding density present within a spot/chamber of the array. FIG. 4B shows a pictogram of a mesh (850), placed in a mesh holder (852) and printed with grid lines (such as exemplary representative gridlines 854A-B) having a thickness/width of 1.5 mm to form chambers (such as exemplary representative chambers 856A-B) having dimensions of 2 mm×2 mm. FIG. 4C shows a pictogram of 2 mm² spots of cell groups (shown as representative cell groups 862A-B) seeded on a substrate (a transparent coated glass slide (860)) using the mesh of FIG. 4B.

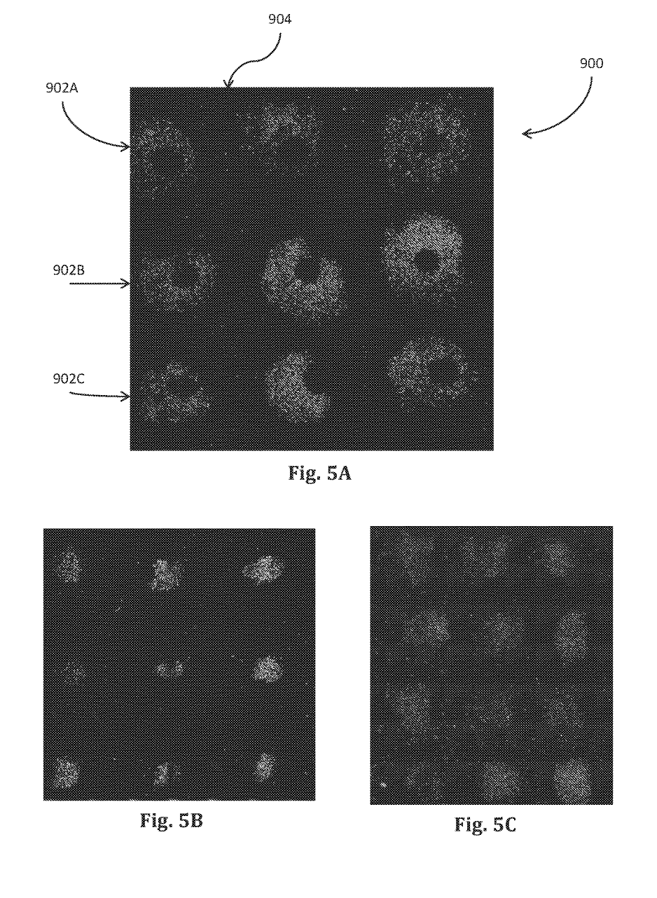

The results presented in FIG. 5A show pictogram of part of a surface of a substrate (shown as slide (900)) showing groups of cells (shown as exemplary groups 902A-C), generated by seeding cell suspension solution through a 100 μm nylon mesh gridded by the hydrophobic polymer. The polymer grid dictated the cluster pattern wherein the cells are spatially separated by separated by cell-free area (for example, 904). Similarly, the results presented in FIGS. 5B-C show pictograms of part of a surface of a substrate seeded with lower amount of cells (FIG. 5B) or higher amount of cells (FIG. 5C), as compared to the amount of cells used in the seeding shown in FIG. 5A. The cells shown in FIG. 5A were seeded manually (i.e., cell suspension was dripped manually) and the cells shown in FIGS. 5B-C were seeded by an automatic dispenser.

### Example 2: Preparing a Nucleic-Acid Array

Exemplified herein is the preparation of a single glass slide (substrate) carrying separable sets of nucleic acid molecules (in the form of DNA) in an array pattern.

According to the example, a nucleic-acid mesh is stretched over a dedicated frame. The mesh in this example is a 41 μm nylon mesh (for example Merck Millipore, cat no. NY4100010). The mesh is patterned by vertical and horizontal 1 mm thick lines of liquid, thermoplastic, polymeric, non-toxic hydrophobic material with 2.5 mm² chambers (printing spaces/spots) confined by the vertical and horizontal hydrophobic lines resulting in a 3.5 mm pitch. The mesh is then baked for a duration of 20 min in an oven pre-heated to 100° C. to solidify the polymer pattern.

The mesh is then aligned over a DNA binding substrate in a dedicated casing. Once alignment of the substrate and the mesh is obtained, multiple sets of mixtures each containing different nucleic acid molecules is dispensed/dripped onto specific predetermined chambers of the gridded mesh. Dispensing may be performed manually or using an automated or semi-automated dispenser.

Additional step of irrigation may be added at this step to promote DNA transfer to the substrate, in which 0.3 ul drops of DNA-free liquid solution are deposited in each chamber.

The mesh is then incubated with the substrate for a period of 20 minutes.

To separate the mesh from the slide, the slide-mesh interface is flooded with a suitable fluid to cancel out capillary forces fastening the mesh to the slide. Then the mesh frame (with the mesh) is gently removed from the substrate, to result with a substrate having an array of sets of nucleic acid molecules.

### Example 3: Preparing an Antibody Array

Exemplified herein is the preparation of single glass slide carrying separable sets of antibodies directed against cell-surface markers in an array pattern.

According to the example, a mesh is stretched over a dedicated frame. The mesh in this examples is a 60 μm nylon mesh (for example Merck Millipore, cat no. NY6000010). The mesh is patterned by vertical and horizontal 1 mm thick lines of liquid, thermoplastic, polymeric, non-toxic hydrophobic material with 1 mm² chambers (printing spaces/spots) confined by the vertical and horizontal hydrophobic lines resulting in a 2 mm pitch. The mesh is then baked for a duration of 20 min in an oven pre-heated to 100° C. to solidify the polymer pattern.

The mesh is then aligned over a hydrogel coated slide (Full Moon Biosystems) which is a glass slide coated by covalently binding material, placed in a dedicated casing. Once alignment of the substrate and the mesh is obtained, multiple different 0.5 mg/ml solutions of different antibodies against different cell surface markers are dispensed/dripped each onto a specific predetermined chambers of the gridded mesh. Dispensing may be performed manually or using an automated or semi-automated dispenser.

Additional step of irrigation may be added at this step to promote antibody transfer to the substrate, in which 0.3 ul drops of PBS solution are deposited in each chamber.

The mesh is then incubated with the substrate for a period of 72 hours at 4° C., in a highly humid environment such that the antibody-free spaces on the slide are blocked.

To separate the mesh from the slide, the slide-mesh interface is flooded with 2-3 ml of PBS solution to cancel out capillary forces fastening the mesh to the slide. Then the mesh frame (with the mesh) is gently removed from the substrate, to result with a substrate having an array of antibodies against cell-surface markers.

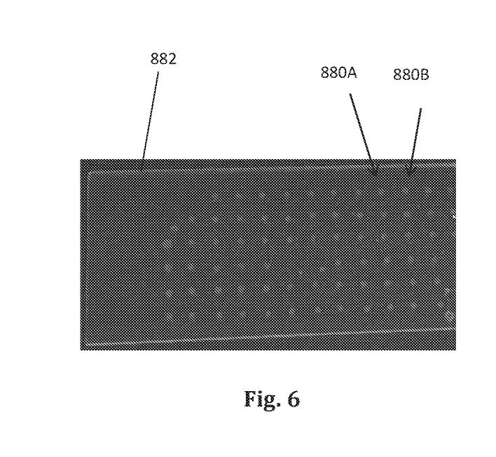

The results are presented in FIG. 6, which shows a pictogram of an exemplary array of antibodies (shown as spots, such as representative spots 880A-B) transferred onto a substrate (shown as glass slide, 882), through a suitable gridded mesh.

1. A system for seeding cells, the system comprising:
a transparent substrate having a cell seeding surface configured for attachment of viable cells;
a seeding mesh having a patterned structure, configured to enable passage of viable cells dispensed onto said seeding surface according to a seeding pattern determined by the patterned structure of said mesh; and
a cell dispenser, configured to provide viable cells onto said mesh.

2. The system of claim 1, wherein said mesh is comprised of a polymeric material.

3. The system of claim 2, wherein the polymeric material comprises nylon, polyester, polyurethane, Polyethylene (PE) polyethylene terephthalate (PET), Polypropylene (PP), Polyvinyl chloride (PVC), Polytetrafluoroethylene (PTFE), Polyvinylidene fluoride (PVDF), Polydimethylsiloxane (PDMS), or combinations thereof.

4. The system of claim 1, wherein the patterned structure comprises a lattice pattern, web pattern, net pattern or honeycomb pattern.

5. The system of claim 1, wherein said mesh is configured to enable homogeneous dispersion of the viable cells onto said seeding surface such that the cell density is substantially uniform within the perimeters of the seeding pattern on said seeding surface.

6. The system of claim 1, wherein said mesh is configured to enable dispersion of viable cells in a plurality of groups, separated from each other at distinct locations on said seeding surface.

7. (canceled)

8. The system of claim 6, wherein said mesh comprises a grid configured to facilitate separation between said cell groups.

9. The system of claim 8, wherein said grid is separate from said seeding mesh and is configured to be mounted on said mesh prior to seeding.

10. The system of claim 8, wherein said grid comprises a non-toxic hydrophobic material.

11. (canceled)

12. The system of claim 1, further comprising a frame for maintaining the orientation of the mesh in alignment with the cell seeding surface.

13. A kit for seeding cells, the kit comprising:
a transparent substrate having a cell seeding surface configured for attachment of viable cells;
a seeding mesh having a patterned structure, configured to allow passage of the viable cells and distribute the viable cells onto said seeding surface according to a seeding pattern determined by the patterned structure of said mesh; and
a frame for maintaining the orientation of the mesh in alignment with the cell seeding surface.

14. The kit of claim 13, wherein said mesh is comprised of a polymeric material.

15. The kit of claim 13, wherein the patterned structure comprises a lattice pattern, web pattern, net pattern or honeycomb pattern.

16. The kit of claim 13, wherein said mesh is configured to enable homogeneous dispersion of said viable cells on said seeding surface such that the cell density is substantially uniform within the perimeters of the seeding pattern on said seeding surface.

17. The kit of claim 13, wherein said mesh is configured to enable dispensing viable cells in multiple groups, separated from each other, at different locations on said seeding surface.

18. The kit of claim 17, wherein said separated groups are homogeneously distributed on said seeding surface.

19. The kit of claim 17, wherein said mesh further comprises a grid configured to facilitate separation between said cell groups according to a predetermined array.

20. The kit of claim 17, wherein said grid is separate from said seeding mesh and is configured to be mounted on said mesh prior to seeding the cells.

21. The kit of claim 17, wherein said grid comprises a hydrophobic material.

22. (canceled)

23. (canceled)

24. A method for seeding cells on a seeding substrate, the method comprising:
providing a cell seeding mesh having a patterned structure;
mounting the seeding mesh on a surface of a seeding substrate;
dispensing viable cells in a physiologically acceptable fluid to the surface of the seeding substrate through the mesh, thereby obtaining multiple viable cells attached to the surface of the substrate according to the pattern of the mesh.

25. The method of claim 24, further comprising providing a fluid layer between the surface of the substrate and the mesh to facilitate passage of the viable cells from the mesh to the substrate.

26. The method of claim 24, further comprising separating the mesh from the surface of the substrate without compromising the seeding pattern and/or the vitality of the cells.

27. The method of claim 24, wherein said mesh comprises a grid structured such that upon dispensing the viable cells to the substrate through the mesh, a plurality of viable cell groups are obtained on the surface of the substrate, such that the cell groups are deposited in an array, determined by the grid on said mesh.

28. The method of claim 24, wherein the grid comprises a hydrophobic non-toxic material.

29. The method of claim 24, further comprising incubating the cells on the substrate.

30.-37. (canceled)
